(19) United States
(12) Patent Application Publication
(10) Pub. No.: US 2015/0359953 A1
(43) Pub. Date: Dec. 17, 2015

(54) DIALYZER COMPRISING A BUNDLE OF HOLLOW FIBERS AND METHOD FOR PRODUCING SUCH HOLLOW FIBER

(71) Applicant: B. BRAUN AVITUM AG, Melsungen (DE)

(72) Inventors: Henrik Wolff, Witzenhausen (DE); Christof Strohhoefer, Kassel (DE)

(21) Appl. No.: 14/725,782

(22) Filed: May 29, 2015

(30) Foreign Application Priority Data
Jun. 12, 2014 (DE) 10 2014 108 230.3

(51) Int. Cl. A61M 1/16; B01D 67/00; B29C 35/16; B01D 71/68; B29C 47/00; B01D 63/02; B01D 69/08

(52) U.S. Cl. CPC A61M 1/16; B01D 63/02; B01D 67/0004; B01D 69/08; B01D 71/68; B29C 47/0023; B29C 35/16; B01D 2319/04; B01D 2323/12; B29K 2081/06

(57) ABSTRACT

A dialysis membrane and a hollow fiber as pre-product as well as a method for producing the hollow fiber are disclosed. The dialysis membrane includes a distribution of the pore sizes which follows an exponential function such as an e-function. The inverse value of the exponential coefficient (K) is at least 30 nm². The dialysis membrane includes at least 50 pores per μm² and the share of a free flow area at a surface of the dialysis membrane amounts to at least 2.5%.

### CROSS REFERENCE TO RELATED APPLICATIONS

This application claims priority to German application DE 10 2014 108 230.3 filed Jun. 12, 2014, the contents of such application being incorporated by reference herein.

### FIELD OF THE INVENTION

The invention relates to a dialyzer comprising a bundle of hollow fibers and a method for producing the same.

### BACKGROUND OF THE INVENTION

The production of hollow fibers and the further processing thereof into membranes for dialysis is basically known, for example from Uhlenbusch-Korwer, I.; Bonnie-Schorn, E.; Grassmann, A. & Vienken, J. (Ed.) “Understanding Membranes and Dialyzers”, Pabst Science Publishers, 2004. Accordingly, the fiber spinning is implemented in six basic process steps. This production process is basically illustrated in FIG. 1.

Hence the first and most important partial process consists in producing a hollow fiber form. This is done in a mixing or annular nozzle in which a precipitating agent flowing in longitudinally central direction into the annular nozzle is injected into a ring (annular flow) of a polymer solution such as polysulfone, so as to axially flow out of the annular nozzle. The second partial process is the actual precipitation of the polymer membrane which is carried out in a tempered water bath axially beneath the nozzle. In the next step the resulting hollow fiber is guided via deflection rollers through a rinsing bath in which it is rinsed to be freed from the residues of the precipitating process so that in the subsequent partial process of drying only the pure hollow fiber is treated. Finally the hollow fibers are wound onto a coil until the number of fibers corresponding to the dialyzer surface to be built up is reached. For filter manufacture fiber bundles are cut out of the hollow fiber wound onto the coil and are processed for mounting in a dialyzer casing.

### DESCRIPTION OF THE RELATED ART

In the state of the art it is especially attempted to optimize the flow and pressure characteristics of a dialyzer with selected membrane parameters so as to achieve an improved medium-molecular clearance.

From U.S. Pat. No. 5,730,712 A in general an apparatus and a method for extracorporeal blood treatment are known. There a flow resistance is provided in a dialyzer so as to partly inhibit the flow of dialysis fluid through the dialyzer. As a result, the pressure in the dialysis fluid above the flow resistance is sufficient to generate a non-linear pressure profile of the dialysis fluid. In this way, large water content can be removed from the blood of a patient by one single dialyzer.

EP 1 406 685 A1 (WO 02/098490) describes a dialyzer having a bundle of plural hollow fibers in a casing. A dialysis fluid flows from an axial inlet of the dialyzer casing through the fiber bundle to an axially opposed outlet of the dialyzer casing, whereas the patient's blood flows outside the hollow fibers axially within the dialyzer casing in accordance with the principle of counter-flow. Within the dialyzer casing the fiber bundle forms a flow channel being in fluid communication with the inlet and consisting of a number of longitudinal grooves provided in the fiber bundle which are spread over the periphery of the fiber bundle.

From EP 1 344 542 A1 a dialyzer is known including an approximately cylindrical casing in which a hollow fiber bundle is provided. The hollow fiber bundle is arranged especially in a heat-shrink tube.

From DE 10 2007 009 208 A1 a hollow fiber, a hollow fiber bundle as well as a filter made thereof and a method for manufacturing the same are known. The hollow fibers include a bottleneck as flow resistance.

From US 2007/0119781 A1 furthermore an apparatus and a method for improved hemodialysis are known. In accordance with this document, one or more nano pore tubes are used as hemodialysis membrane. These tubes are adapted to be manufactured with a nano pore wall structure having a mean pore diameter of approx. between 5 nm and 10 nm. In the state of the art the pore size of a dialysis membrane is established indirectly by establishing screening characteristics of the membrane. It is assumed in this context that the pore size follows a quasi-normal distribution.

The hollow fiber membranes empirically developed in the state of the art inter alia show the following drawbacks, however;

1. The previously known hollow fiber membranes are not optimized to being applied as dialyzer for extracorporeal blood treatment. The pore diameter thereof is less than 6 nm with pore sizes below 30 nm² for high-flux dialyzers, and the pore diameter is less than 10 nm with pore sizes below 80 nm² for high-cutoff dialyzers. In accordance with the present invention, it has turned out that these values are not optimal for the purification of medium-molecular substances.
2. During judgment (performance assessment) of hollow fiber membranes furthermore in the state of the art neither the pore density nor the free flow area of the membrane is taken into account.

Comparing dialyzers including polysulfone hollow fiber membranes and otherwise having equal parameters, the following is resulting:

| | Dialyzer 1 | Dialyzer 2 | Dialyzer 3 |
|---|---|---|---|
| Pore size nm² | 87 | 164 | 183 |
| Pores/μm² | 62 | 126 | 122 |
| Free flow area/% | 1.32 | 2.84 | 3.21 |
| Clearance | 100 | 127 | 140 |
| cytochrome C/ | | | |
| ml/min with blood | | | |
| flow 300 | | | |

It is clearly visible here that the embodiments according to the exemplified dialyzers 1 to 3 having optimized membrane parameters promote the transport of cytochrome C; i.e., the dialyzer clearance generally increases along with the increase in the free flow area.

However, in accordance with the invention in dialysis there is a need for membranes having a pore number and a pore size distribution by which a low flow resistance for the transport of substances of more than 500 Da and less than 60 kDa can be achieved and at the same time a preferably high flow resistance is ensured for the transport of substances of more than 60 kDa.

### SUMMARY OF THE INVENTION

Therefore it is an object of the invention to provide a hollow fiber membrane as well as a method for producing the same including quality control by which the pore structure and, respectively, the pore characteristic are better adjusted to the requirements of a hollow fiber membrane filter for dialysis purposes (especially extracorporeal blood treatment).

This object is achieved by a dialyzer comprising hollow fibers and a method for producing such hollow fibers.

On principle, the invention relates to the following approach:

During production of the hollow fiber according to aspects of the invention manufacturing parameters selected in accordance with the invention such as the ratio of a hydrophobic polymer to a hydrophilic polymer are adjusted or set preferably empirically (or according to the “trial and error” method) so that such adjustment/setting will result in a hollow fiber having characteristics according to aspects of the invention (optimized for the use as a dialysis membrane). The characteristics of the dialysis membrane preferably are
the type of pore size distribution and/or
the pore size and/or
the pore density in 1/mm² and/or
the free flow area of the membrane in % of the membrane surface area.

The quality of the membrane can be checked for its properties directly after producing/spinning the hollow fiber so that in this way the selected manufacturing parameters (variables influencing the membrane properties) can be appropriately controlled.

The method according to aspects of the invention for producing a hollow fiber from a polymer solution and a precipitating agent consequently comprises at least the following steps preferably in the given order:

a) (pre-)setting selected manufacturing parameters preferably of a polymer solution (polymer mixture),
b) producing a hollow fiber form in an (annular) nozzle in which a precipitating agent having a predetermined concentration is injected into a ring made of the polymer solution,
c) precipitating the hollow fiber/polymer membrane in a tempered water bath beneath the nozzle,
d) guiding the hollow fiber via deflecting rollers through a rinsing bath,
e) rinsing the hollow fiber to remove residues of the precipitating process in the rinsing bath,
f) drying the pure hollow fiber,
g) winding the hollow fiber onto a coil until the number of hollow fibers corresponding to the dialyzer surface is reached,
h) checking the current hollow fiber characteristic (especially pore size and pore density as well as preferably pore size distribution and free flow area),
i) comparing the current hollow fiber characteristic to a desired hollow fiber characteristic (which is optimal to the extracorporeal blood treatment) and, respectively, a rated range of the hollow fiber characteristic and, if required, readjusting the selected manufacturing parameters, preferably the adjustment of the polymer solution, until the desired hollow fiber characteristic is approached/reached or the current hollow fiber characteristic is within the rated range of the hollow fiber characteristic.

Of preference, the polymer includes polysulfone as hydrophobic component for producing the hollow fiber. In a further preferred embodiment of the method the polymer additionally comprises polyvinylpyrrolidone (PVP) as hydrophilic component for producing the hollow fiber.

Furthermore layers that modify the characteristic of the fiber can be applied. In this way, for example the hemocompatibility of the hollow fiber can be improved.

Especially the ratio of the hydrophobic polymer to the hydrophilic polymer is set to a predetermined/analytically defined value.

In another preferred embodiment of the method the hollow fiber is wound onto a coil in plural layers or windings.

Preferably, the desired pore size to be reached/approached via the method amounts to at least 30 nm², preferably at least 80 nm², on the inside of the membrane, i.e. the part of the membrane which is in contact with blood. Alternatively, the desired pore size defined in this way is within a range of 30-80 nm².

In a further preferred manner, the pore density to be reached/approached amounts to at least 50 pores/μm².

Of further preference, the free flow area amounts to at least 2.5% of the entire membrane surface area (measured “blood contact area” of the membrane).

Preferably, as a further step before incorporating a hollow fiber bundle combined of the hollow fibers into a dialyzer casing (corresponding to step h)), the method comprises the step of determining the pore size of the membrane and optionally further parameters of an inner face of the hollow fiber by an electron microscope.

Especially, the definition of the pore size of the membrane and optionally of further parameters of an inner face of the hollow fiber by an electron microscope comprises the following preparatory steps:
quick-freezing a hollow fiber preferably in liquid nitrogen,
breaking the quick-frozen hollow fiber for exposing the inner face of the hollow fiber
   and
aligning the hollow fiber with an object carrier so that an electron beam of the electron microscope is incident on the inside.

The method step h) preferably comprises the following sub-steps for analysis/inspection of the manufactured product:
applying the detected pore size to a histogram and adapting an e-function to the distribution of the pore size,
establishing a number of pores for each μm² and a free flow area of the membrane as a function of the sum of all pore sizes and a membrane surface.

Preferably the hollow fiber/dialysis membrane according to aspects of the invention includes the feature that the distribution of the pore sizes follows an exponential function and, in particular, an e-function. Especially the inverse value of the exponential coefficient (K) of said e-function amounts to at least 30 nm² and especially to at least 80 nm².

The hollow fiber/dialysis membrane of the present invention preferably includes at least 50 pores per μm².

More preferably, in the hollow fiber/dialysis membrane of the present invention the share of a free flow area in a surface of the dialysis membrane amounts to at least 2.5%.

Inter alia, the invention offers the following advantages:
An increased efficiency of the medium-molecular substance transport is achieved.
Equally, an optimized quality of treatment can be ensured for the patient.
In total, the purification of medium-molecular substances between 500 Da and 60 kDa is optimized by the new pore structure on the inner face of the hollow fiber membrane.

### BRIEF DESCRIPTION OF THE DRAWINGS

Hereinafter the present invention will be illustrated in detail by way of a preferred embodiment with reference to the accompanying figures.

FIG. 1 shows the basic/functional structure of an apparatus for the production of hollow fibers suited for being incorporated in dialyzers,

FIG. 2 shows the picture of a hollow fiber/membrane according to aspects of the invention by using an electron microscope,

FIG. 3 shows the mathematical processing of the picture according to FIG. 2 for representing the pores formed in the membrane as well as the pore distribution and

FIG. 4 shows a histogram drafted from the representation according to FIG. 2 including the pore size distribution.

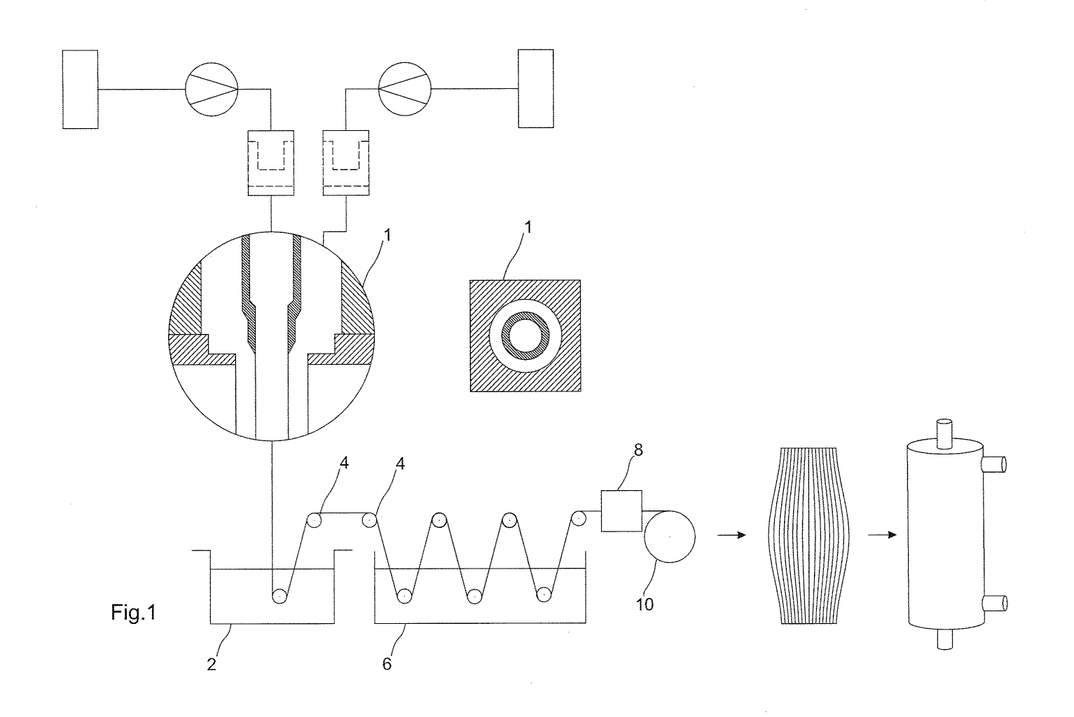

### DETAILED DESCRIPTION OF THE PREFERRED EMBODIMENTS

The method for producing the hollow fiber according to aspects of the invention from a polymer solution and a precipitating agent can be split into the following steps.

Firstly, a hollow fiber form is produced in a nozzle 1 by injecting in the nozzle 1 the precipitating agent at a predetermined concentration into a ring of a selected (pre-adjusted) polymer solution. Subsequently, the hollow fiber/polymer membrane is precipitated in a tempered water bath 2 below (downstream of) the nozzle 1. Via deflection rollers 4 the hollow fiber is guided through a rinsing bath 6 where it is purified from residues from the preceding process steps. This is followed by a rinsing operation in which the hollow fiber is freed from residues of the precipitating process in the rinsing bath. Finally the now purified hollow fiber is dried in a dryer 8 and is wound onto a coil 10. The winding operation is carried out until the number of hollow fibers corresponding to the dialyzer surface is reached. So far the production method is in conformity with the state of the art.

The polymer used for producing the hollow fiber preferably is polysulfone is as the hydrophobic component, and polyvinylpyrrolidone (PVP) is preferably used as the hydrophilic component of the polymer. The ratio of the two components is initially set to a predetermined value (empirical value).

A dialysis membrane is manufactured from the hollow fiber produced in this way by the following steps:

A hollow fiber bundle (not shown in detail as it is known from the state of the art) is cut out of the hollow fiber wound onto the coil 10. The cut-out hollow fiber bundle is incorporated in a dialyzer casing (equally known from the state of the art and therefore not shown in detail) and serves as dialysis filter.

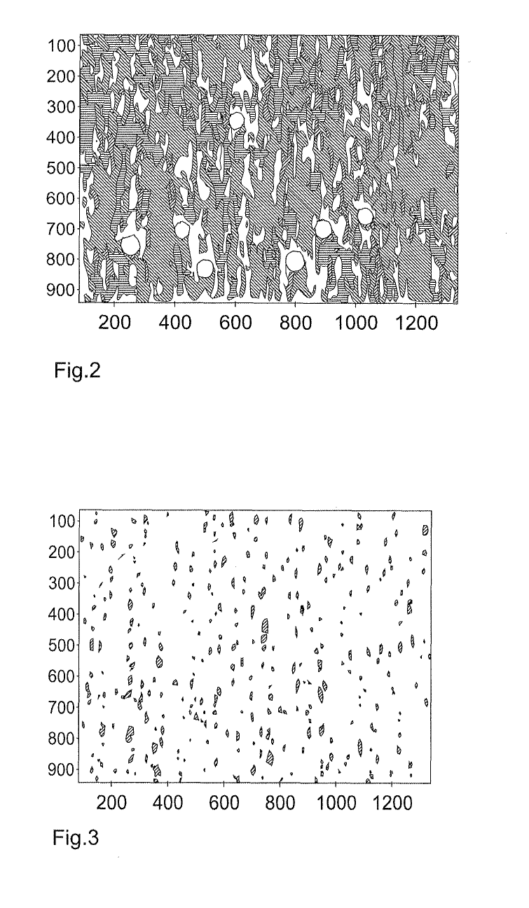

In order to safeguard the properties/characteristics of the dialysis membrane, individual parameters, preferably the pore size, or the pore size distribution, of the hollow fiber/membrane is determined and optionally further parameters of the inner face of the hollow fiber are examined by an electron microscope. In this respect, the accompanying FIGS. 2 to 4 are referred to.

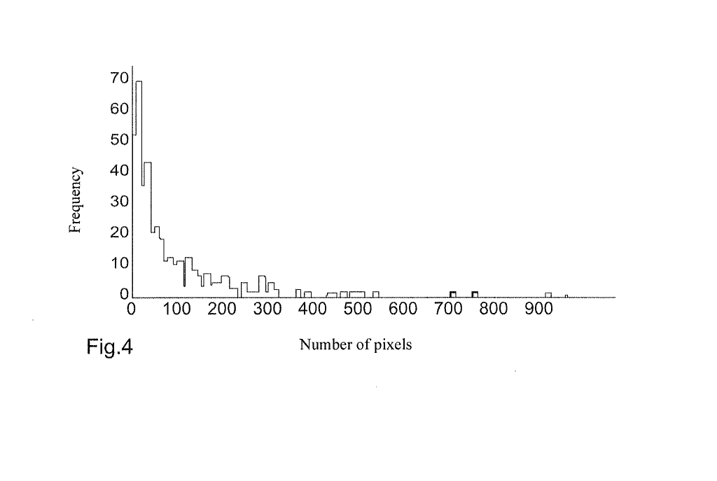

Accordingly, the hollow fibers are detected image-wise by using an electron microscope, wherefrom a surface illustration of the hollow fiber according to FIG. 2, for example, is resulting. This illustration then is subjected to a mathematical processing procedure from which a black-and-white representation approximately according to FIG. 3 is resulting in which only the pores are shown. In this black-and-white representation the pore size as well as the (pore) number thereof can be determined, e.g., by means of pixel numbers or by means of the scale of the picture. Herefrom a histogram according to FIG. 4 can be established having the pore size distribution by applying for example the “frequency” against the “number of pixels”, wherein alternatively the “number of pixels” could also be replaced by the surface area (nm²) and could be appropriately converted, as a matter of course.

In the state of the art verification of these parameters of the dialysis membrane has been implemented not at all or only to a restricted extent/indirectly so far. Therefore in the state of the art only corresponding assumptions have been made about the distribution of the pores in the membrane. Especially, in a simplified manner, a quasi-normal distribution for the pore sizes has been assumed, as the direct influence thereof on the suitability of the hollow fiber for particular purposes (extracorporeal blood treatment) has not been detected or has been underestimated. The pore size therefore was assessed only based on conclusions from the screening characteristics of the membrane. It was not directly measured.

Rather, in the state of the art, the membrane has been characterized with the screening characteristics of the membrane, namely, by means of the size of the molecules allowed to pass the membrane.

However, according to aspects of the invention, for the judgment of the produced membrane as to quality and thus for adjusting the hollow fiber production process the pore size of the hollow fiber/membrane is preferably examined by electron-microscopic measuring technology. The characteristic parameters of the inner faces of the fiber can be established by the electron microscope. The direct electron-microscopic visualizing of the pores is at the resolution limit of the current technology and provides substantiated measuring results as a basis of the determination of the characteristic/quality of the hollow fiber and, where appropriate, of the re-adjustment of the manufacturing parameters.

For sample preparation individual fibers are quick-frozen in liquid nitrogen. Subsequently the fibers are broken so as to take pictures of the surface of the fiber inside. Finally, the fiber is aligned on an object carrier so that the electron beam can be directed to the inner face of the fibers.

Preferably the scanning electron microscope pictures are taken by a scanning electron microscope which is operated, for example, at an accelerating voltage of 3 kV and permits a 50,000 fold magnification. The sample preparation is performed, as afore-mentioned, by quick-freezing of individual hollow fibers in liquid nitrogen, breaking the hollow fibers for exposing the inner face of the hollow fiber so that pictures of the surface of the fiber inside can be taken, and aligning the hollow fiber on an object carrier so that an electron beam of the electron microscope is incident on the inside.

The distribution of the pore size is shown in a histogram. The pore size distribution in the histogram can be described, in accordance with the invention, again by an e-function, wherein a characteristic parameter of the e-function is used, namely the inverse value of the exponential coefficient (K). The latter can be easily established, as at this point an e-function of the type f(x)=A*ê(−(K)*x) adopts the value {(1/e)*A} (wherein A=maximum value).

Furthermore, the number of pores per μm² can be established and the free flow area of the membrane can be determined as a function of the sum of all pore sizes and a membrane surface. The pore density is the number of the pores per μm² and the free flow area puts the sum of all pore sizes in a proportion to the measured surface.

The hollow fiber/dialysis membrane produced in this way exhibits an exponential function and especially an e-function during distribution of the pore sizes. As particularly suited hollow fibers/dialysis membranes those are selected in which the inverse value of the exponential coefficient (K) of the e-function amounts to at least 30 nm² and especially to at least 80 nm². The pore density desired especially is at least 50 pores per μm² and the share of the free flow area in the total surface area of the dialysis membrane amounts to at least 2.5%.

Summing up, the membrane according to aspects of the invention comprises the following characteristics:
I. The pore size distribution follows an exponential function.
II. The inverse value of the exponential coefficient (K) amounts to at least 30 nm² in the case of high-flux dialyzers and preferably to at least 80 nm².
III. The pore density amounts to at least 50 pores/μm².
IV. The free flow area amounts to at least 2.5%.

Hence the invention relates to a dialysis membrane as well as a hollow fiber as pre-product and a method for producing the hollow fiber. The hollow fiber/dialysis membrane according to aspects of the invention includes a distribution of the pore sizes following an exponential function and especially an e-function. The inverse value of the exponential coefficient (K) of the e-function amounts to at least 30 nm² and especially to at least 80 nm². The hollow fiber/dialysis membrane includes at least 50 pores per μm² and the share of a free flow area at a surface of the hollow fiber/dialysis membrane amounts to at least 2.5%.

1-11. (canceled)

12. A dialyzer for extracorporeal blood treatment of a patient comprising:
a dialyzer casing; and
a bundle of hollow fibers accommodated by the casing, each hollow fiber thereof including pores for the passage of substances being at most medium-molecular, wherein the distribution of the pore sizes at an inner surface of the hollow fiber follows an exponential function and wherein the inverse value of an exponential coefficient (K) is at least 30 nm².

13. The dialyzer according to the preamble of claim 12, wherein the pore size is at least 30 nm².

14. The dialyzer according to claim 12, wherein the hollow fiber includes at least 50 pores per μm².

15. The dialyzer according to claim 12, wherein a share of a free flow area at the inner surface or a blood contact surface of the hollow fiber amounts to at least 2.5%.

16. The dialyzer according to claim 12, wherein the exponential function is an e-function.

17. The dialyzer according to claim 12, wherein the exponential coefficient (K) is at least 80 nm².

18. The dialyzer according to claim 13, wherein the pore size is at least 80 nm².

19. A method for producing a hollow fiber of a polymer solution and a precipitating agent comprising the steps of:
a) pre-setting selected manufacturing parameters, including parameters of the polymer solution;
b) producing a hollow fiber with a nozzle in which a precipitating agent is injected at a predetermined concentration into a ring made of the polymer solution;
c) precipitating the hollow fiber in a tempered water bath;
d) guiding the hollow fiber through a rinsing bath;
e) rinsing the hollow fiber to remove residues of the precipitating in the rinsing bath;
f) drying the hollow fiber;
g) winding the hollow fiber onto a coil;
h) checking current hollow fiber characteristic, the characteristic including pore size and pore density;
i) comparing the current hollow fiber characteristic to a desired hollow fiber characteristic for the extracorporeal blood treatment and a rated range of the hollow fiber characteristic; and
j) re-adjusting the selected manufacturing parameters until the current hollow fiber characteristic is within a predefined range of the desired hollow fiber characteristic or the current hollow fiber characteristic is within the rated range of the hollow fiber characteristic.

20. The method according to claim 19, wherein the polymer solution for producing the hollow fiber comprises polysulfone as hydrophobic component.

21. The method according to claim 20, wherein the polymer solution for producing the hollow fiber further comprises polyvinylpyrrolidone (PVP) as hydrophilic component.

22. The method according to claim 21, wherein a proportion of the hydrophobic polymer to the hydrophilic polymer is set to at least one of a predetermined or analytically defined value as the manufacturing parameter.

23. The method according to claim 19, further comprising the step of:
winding the hollow fiber in plural layers on the coil.

24. The method according to claim 19, further comprising the step of:
determining the pore size of the hollow fiber.

25. The method according to claim 24, wherein determining the pore size of the hollow fiber comprises:
quick-freezing the hollow fiber in liquid nitrogen;
breaking the hollow fiber to expose the inner surface of the hollow fiber; and
aligning the hollow fiber on an object carrier so that an electron beam of an electron microscope is incident on the inner surface.

26. The method according to claim 19, further comprising the steps of:
a) applying the pore size to a histogram and adapting an exponential function to the distribution of the pore size; and
b) establishing a number of pores per pmt and a free flow area of the inner surface of the hollow fiber as a function of a sum of all pore sizes and the inner surface.

27. The method of claim 26, wherein the exponential function is an e-function.

28. The method of claim 19, wherein the guiding is performed with deflection rollers.

29. The method of claim 19 wherein the characteristic further includes pore size distribution and free flow area.
